SEQUENCE LISTING (3 sequences)

SEQ ID NO: 1 - protein, 42 aa, Homo sapiens [sequence omitted]

SEQ ID NO: 2 - protein, 12 aa, Homo sapiens
YNVHHQKLVFFA

SEQ ID NO: 3 - protein, 12 aa, Homo sapiens
YEVQHQKLVFFA

(19) United States
(12) Patent Application Publication
(10) Pub. No.: US 2005/0164361 A1
(43) Pub. Date: Jul. 28, 2005

(54) Self-assembling-peptide-based structures and processes for controlling the self-assembly of such structures

(76) Inventors: David Lynn, Atlanta, GA (US); Vincent Conticello, Decatur, GA (US); David M Morgan, Dallas, TX (US); Jijun Dong, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/508,586

(22) PCT Filed: Mar. 24, 2003
(86) PCT No.: PCT/US03/09229

Related U.S. Application Data
(60) Provisional application No. 60/366,826, filed on Mar. 22, 2002.
(60) Provisional application No. 60/420,746, filed on Oct. 23, 2002.

(30) Foreign Application Priority Data
Mar. 21, 2003 (US) 60456641

(51) Int. Cl. C12N 11/04; C12N 9/64

(52) U.S. Cl. 435/182; 435/226

(57) ABSTRACT

The thermodynamics of self-assembling peptides may be altered to produce different morphologies. By altering environmental factors, initiation and propagation of self-assembly processes may be altered, thereby consequently altering the morphology of the resultant structure.

### CROSS REFERENCE TO RELATED APPLICATIONS

This application claims the benefit of U.S. provisional patent applications having Ser. No. 60/366,826, filed on Mar. 22, 2002, Ser. No. 60/420,746, filed on Oct. 23, 2003, and Express Mail mailing label number EV269328445US, filed on Mar. 21, 2003, which are incorporated herein by reference in their entireties.

### FIELD OF INVENTION

The present invention relates generally to peptides and, more particularly, to self-assembling-peptide-based structures and processes for controlling the self-assembly of such structures.

### BACKGROUND

Nanotechnology has recently become of great interest for a variety of reasons. For example, nanostructures may be used to generate devices at a molecular level, thereby permitting molecular-level probing. Specifically, it has been suggested that fibrils can be used for connectors, wires, and actuators. Additionally, it has been suggested that nanotubes may be used as miniature pipettes for introducing small proteins into biological or other systems.

Nanotubes may be generated through carefully controlled high-energy kinetic processes, in which graphite-based structures (e.g., “bucky” tubes) are formed at extremely high temperatures. However, the outcome of these kinetic processes is often difficult to predict, and the resulting structure tends to be heterogeneous.

Given the relatively unpredictable kinetic processes related to graphite-based structures, a need exists in the industry for a robust nanostructure that can be created homogeneously and without the need for complicated kinetic processes.

### SUMMARY

The present disclosure provides self-assembling-peptide-based structures and processes for controlling the self-assembly of such structures.

Briefly described, in architecture, one embodiment is a fibril or nanotube structure generated as a result of controlling changes in the environment during a self-assembly process.

The present disclosure also provides processes for controlling the self-assembly of self-assembling-peptide-based structures.

In this regard, one embodiment of the method comprises the steps of placing a self-assembling peptide in a controlled environment, and controlling the initiation and propagation of a self-assembly process by controlling the environment.

Other systems, methods, features, and advantages will be or become apparent to one with skill in the art upon examination of the following drawings and detailed description. It is intended that all such additional systems, methods, features, and advantages be included within this description.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a diagram illustrating the structure of an example amyloid fibril.

FIG. 2 is a diagram illustrating laminated β-sheets within the amyloid fibril of FIG. 1.

FIG. 3 is a diagram illustrating, in greater detail, two adjacent β-sheets of FIG. 2 and the positions of the side chains.

FIG. 4 is a diagram illustrating potential metal ion binding sites between two β-strands along the β-sheet within the laminated structure of FIG. 2.

FIG. 5A is a graph showing normalized rate of fibril formation as a function of metal ion content for Aβ(10-21) (amino acid residues 10-21 of SEQ ID NO: 1).

FIG. 5B is a graph showing normalized rate of fibril formation as a function of metal ion content for Aβ(10-21)H13Q (SEQ ID NO: 3).

FIG. 6 is a diagram showing long homogeneous fibers that are formed in the absence of metal ions.

FIG. 7 is a diagram showing numerous short fibers that are formed in the presence of metal ions.

FIG. 8 is a diagram illustrating one embodiment of a structure as a rectangular bilayer that is formed as an aggregate of fibril segments.

FIG. 9 is a graph showing progression of mean residue ellipticity over time, which is indicative of the structures being formed over time.

FIG. 10 is a diagram illustrating a top view of an example nanotube formed from amyloid fibrils.

FIG. 11 is an exploded view of a section of the nanotube of FIG. 10.

### DETAILED DESCRIPTION OF THE PREFERRED EMBODIMENTS

Reference is now made in detail to the description of the embodiments as illustrated in the drawings. While several embodiments are described in connection with these drawings, there is no intent to limit the invention to the embodiment or embodiments disclosed herein. On the contrary, the intent is to cover all alternatives, modifications, and equivalents.

It is known that certain peptides self-assemble into various peptide-based structures through thermodynamic processes. By manipulating the environment of these self-assembling peptides, the nucleation and propagation of the self-assembly process may be controlled. Consequently, manipulating environmental factors may predictably control the morphology of the self-assembled structures. The use of self-assembling peptides is advantageous because, unlike graphite-based structures that require high-energy kinetic processes, these self-assembling peptides organize themselves through thermodynamic processes. In this regard, the resulting peptide-based structure requires very little overhead.

Another advantage in using self-assembling peptides is that, unlike lipid-based structures that are relatively loosely structured, the architectural integrity of the resulting peptide-based structure is fairly robust due to the hydrogen (H) bonds along the backbone that define the structure. In this regard, peptide-based structures enjoy a distinct advantage over both lipid-based structures and graphite-based structures.

The description below provides methods for controlling nucleation and propagation in forming peptide-based structures, thereby controlling the morphology of the resulting peptide-based structure. In a general sense, self-assembly provides a controlled environment where well-defined and homogeneous structures form. The following description merely outlines example architectures that may be formed as a result of controlling environmental factors that affect the nucleation and propagation of formation.

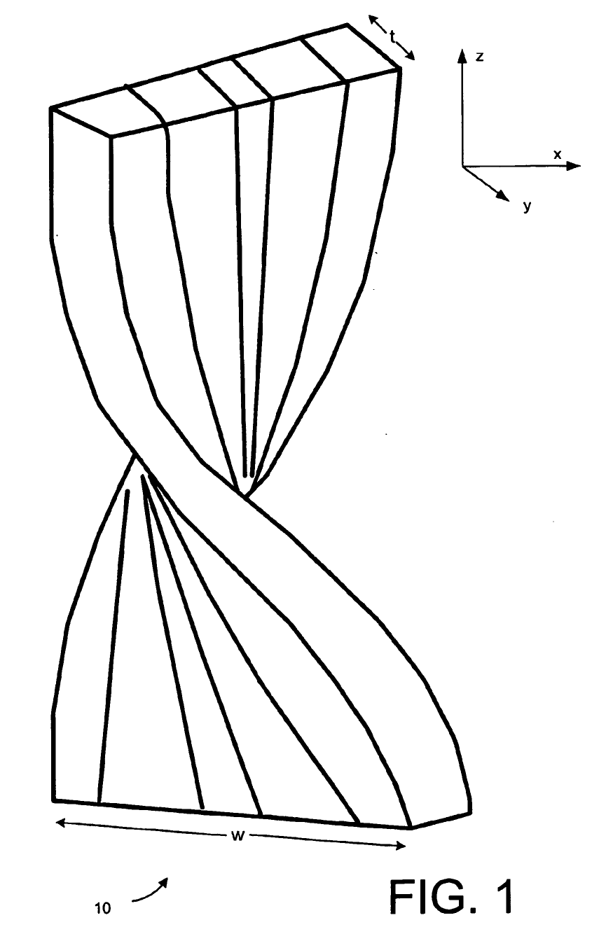

FIG. 1 is a diagram illustrating the structure of an example amyloid fibril 10. Specifically, FIG. 1 shows an Aβ(10-35) (amino acid residues 10-35 of SEQ ID NO: 1) fibril having multiple β-sheets that are laminated and, in the aggregate, form the fibril 10. As shown in FIG. 1, hydrogen bonding (H-bonding) between adjacent residues results in a lamination of multiple sheets. The H-bonding further stabilizes the structure of the fibril 10. Depending on the length of the segments that comprise the fibril 10, the effect of the H-bonding between these segments may differ. Since the H-bonding contributes to the curvature of the fibril 10 as shown in FIG. 1, the length of the segments may further contribute to the degree of curvature of the fibril 10, thereby further affecting the morphology of structures that can be formed from the fibrils. While the exact mechanism is still not fully understood, it is clear that topology is correlated to the length of the fiber segments. This is evidenced by the different resulting topologies from Aβ(10-35) (amino acid residues 10-35 of SEQ ID NO: 1) and Aβ(10-22) (amino acid residues 16-22 of SEQ ID NO: 1), which are described in greater detail in the paper “Exploiting Amyloid Fibril Lamination for Nanotube Self-Assembly,” by Lu et al., which is set forth fully in U.S. provisional patent application having Express Mail mailing label number EV269328445US, filed on Mar. 21, 2003. Given this observation, it is clear that, in one embodiment, the architecture of self-assembled structures may be altered by modifying the length of the component fiber segments.

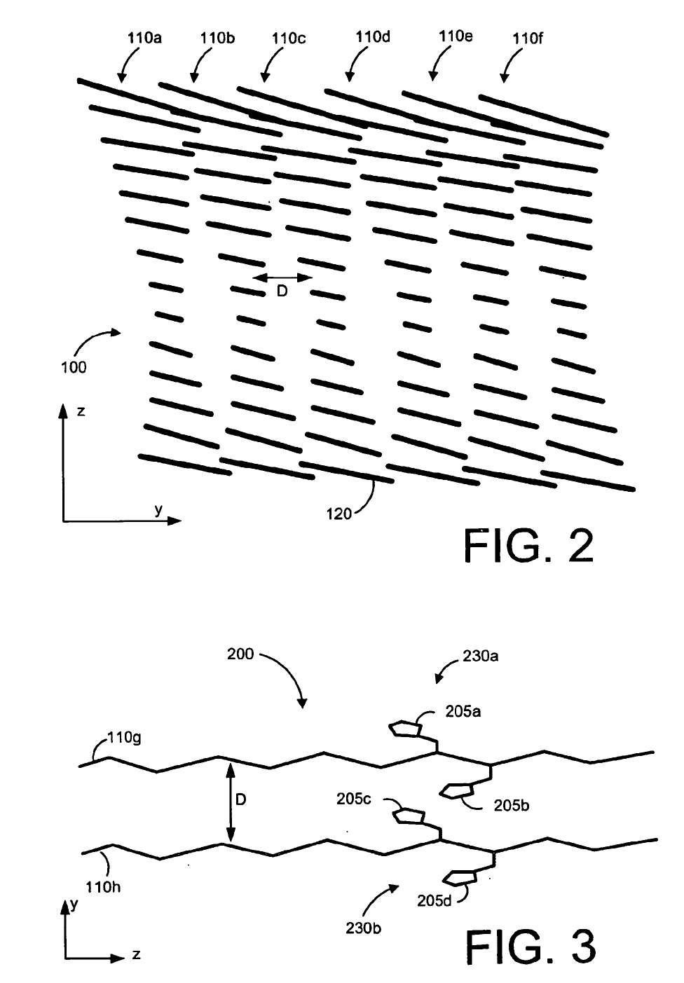

The component structure of the amyloid fibril 10 is discussed in greater detail in FIGS. 2 and 3, which are diagrams illustrating laminated β-sheets 100 within the amyloid fibril 10 of FIG. 1. As shown in FIG. 2, the β-sheets 100a . . . 100f in Aβ(10-35) (amino acid residues 10-35 of SEQ ID NO: 1) align parallel to each other due to the H-bonding. These sheets, as shown in FIG. 3, have a relatively fixed sheet separation (D) that is governed by the attractive and repulsive forces resulting from the formation of H-bonds along the backbone of the fiber segments. Additionally, residues 205a . . . 205d along the β-sheets 100g, 100h are arranged in a fairly organized manner due to these attractive and repulsive forces. For reasons provided below, these residues 205a . . . 205d may provide binding sites for substances such as, for example, metal ions, which affect the nucleation and propagation of fibril formation.

Changing the acidity (pH) of the environment (e.g., between approximately 2 and approximately 7.5) results in an alteration of the attractive and repulsive forces. Since, as described above, the self-assembling-peptide-based structure is likely based on H-bonds formed between the component segments, changes in the pH, which is effectively an alteration of the H⁺ content, result in morphological changes. Typically, the rate of formation decreases at lower pH and increases at higher pH. In this regard, in another embodiment, the resulting morphology may be changed by altering the pH of the environment in which the self-assembly process takes place. As non-limiting examples, a pH of approximately 2.0 may provide a relatively homogeneous self-assembled structure while a more neutral pH (e.g., approximately 7.0 to approximately 7.4) may provide a fairly heterogeneous self-assembled structure. The changes in morphology can be seen by comparing the different morphologies presented in the papers “Structure of the β-Amyloid(10-35) (amino acid residues 10-35 of SEQ ID NO: 1) Fibril,” by Burkoth et al., which is fully set forth in U.S. provisional patent application having Ser. No. 60/366,826, filed on Mar. 22, 2002, “Metal Switch for Amyloid Formation: Insight into the Structure of the Nucleus,” by Morgan et al., which is fully set forth in U.S. provisional patent application 60/420,746, filed on Oct. 23, 2003, and “Exploiting Amyloid Fibril Lamination for Nanotube Self-Assembly.”

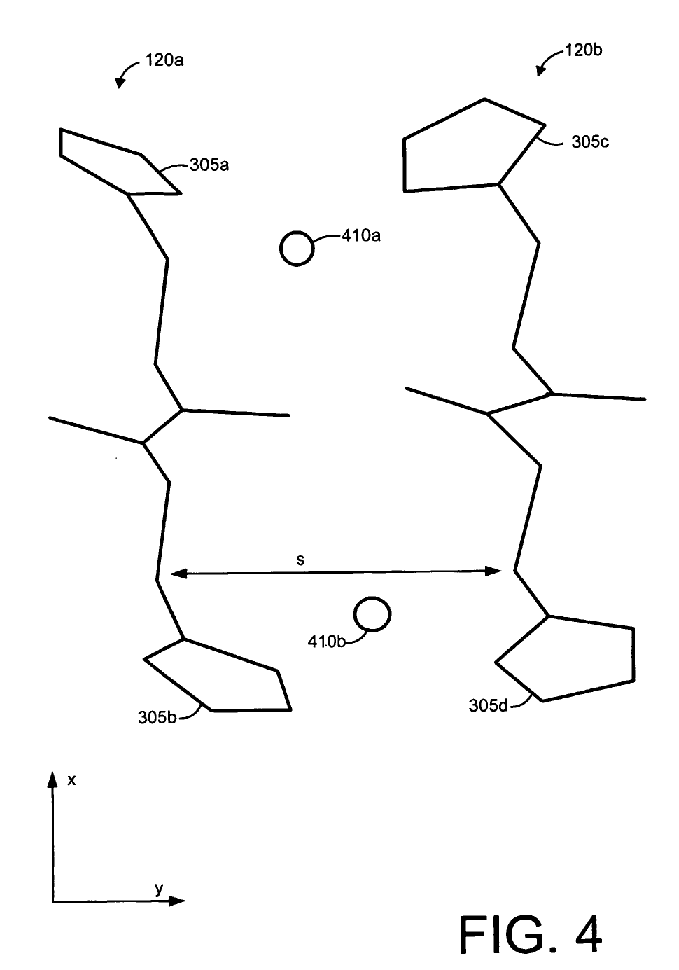

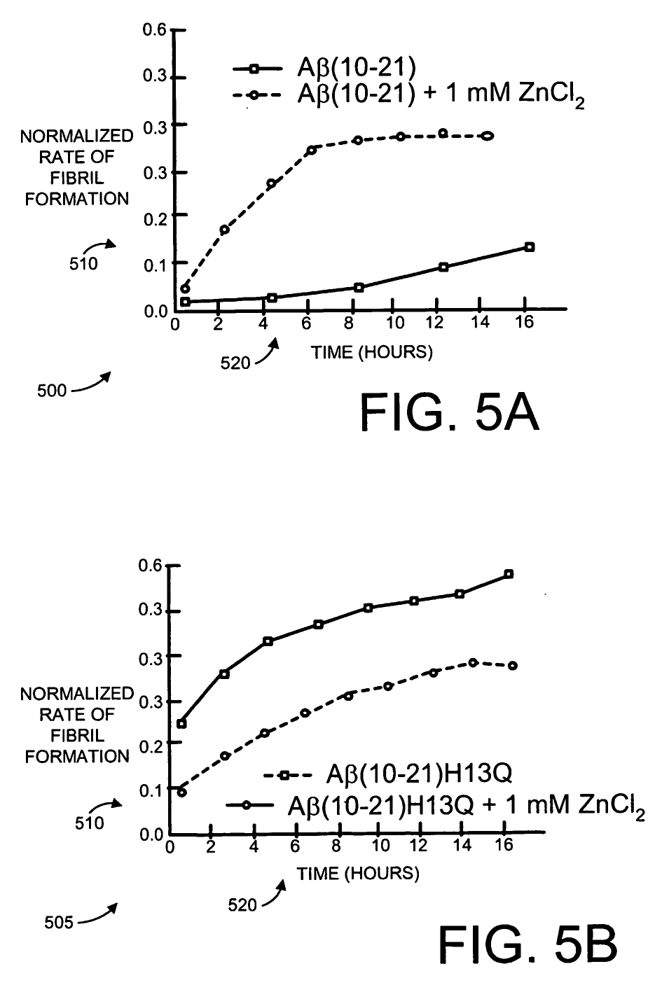

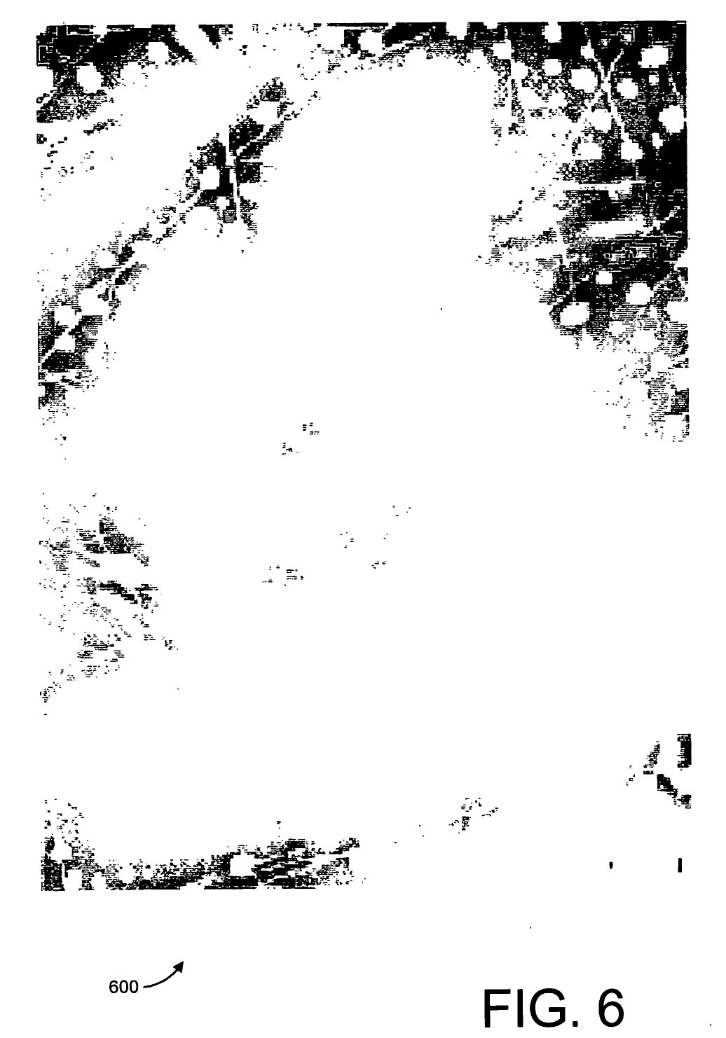

FIG. 4 is a diagram illustrating potential metal ion binding sites between two β-strands in the laminated structure of FIG. 2. As shown in FIG. 4, the β-strands 120a, 120b have residues 305a . . . 305d that are arranged in a particular configuration as a result of the H-bonds within and between the β-strands 120a, 120b. Consequently, the resulting configuration produces potential binding sites in close proximity to adjacent residues 305a, 305c between two β-strands 120a, 120b that may bind to a metal ion 410a. The changes in attractive and repulsive forces due to the metal ion may further contribute to the morphology of the resulting peptide-based structure. Additionally, the presence of the metal ions may facilitate the self-assembly process by pre-organizing the component segments. Thus, in another embodiment, the architecture of self-assembled structures may be altered by modifying metal content in the environment, thereby affecting how the self-assembling peptides interact with each other in forming the resulting self-assembled structure. Details related to the nucleation and propagation of self-assembly are described with reference to FIGS. 5A and 5B, and are also described in the paper “Metal Switch for Amyloid Formation: Insight into the Structure of the Nucleus.”

FIG. 5A is a graph 500 showing normalized rate of fibril formation as a function of metal ion content for Aβ(10-21) (amino acid residues 10-21 of SEQ ID NO: 1). Specifically, FIG. 5A plots the normalized rate of fibril formation on the y-axis 510 and the time on the x-axis 520 of the graph 500. In the example of FIG. 5A, the metal ion is a zinc ion (Zn⁺²) which is introduced into the environment of the self-assembling peptide as zinc chloride (ZnCl₂). As shown in FIG. 5A, Aβ(10-21) (amino acid residues 10-21 of SEQ ID NO: 1) self-assembles at a relatively slow rate in the absence of ZnCl₂. Comparatively, in the presence of ZnCl₂, Aβ(10-21) (amino acid residues 10-21 of SEQ ID NO: 1) self-assembles at a much higher rate to form the resulting self-assembled structure.

FIG. 5B is a graph 505 showing normalized rate of fibril formation as a function of metal ion content for Aβ(10-21)H13Q (SEQ ID NO: 3), which is Aβ(10-21) having a modified amino acid residue 13. Specifically, FIG. 5B plots the normalized rate of fibril formation on the y-axis 510 and the time on the x-axis 520 of the graph 505. Again, Zn⁺² is used as the metal ion. As shown in FIG. 5B, Aβ(10-21)H13Q (SEQ ID NO: 3) shows a greater propensity toward amyloid formation even in the absence of the zinc ion. Thus, for Aβ(10-21)H13Q (SEQ ID NO: 3), the nucleation period was greatly shortened so as to be almost undetected in FIG. 5B. As shown in FIG. 5B, the rate of self-assembly is comparatively higher for Aβ(10-21)H13Q (SEQ ID NO: 3) in the presence of ZnCl₂ than in the absence of ZnCl₂. While not shown in FIG. 5A or 5B, the nucleation (or activation) of self-assembly is inhibited by the introduction of copper (Cu⁺²), rather than Zn⁺², into the environment of the self-assembling peptide.

Since the ramifications of FIGS. 5A and 5B are discussed in greater detail in the paper “Metal Switch for Amyloid Formation: Insight into the Structure of the Nucleus,” only a truncated discussion of the effects of ZnCl₂ on Aβ(10-21) (amino acid residues 10-21 of SEQ ID NO: 1) is discussed here. However, as evidenced by the two graphs 500, 505, it should be appreciated that, in a more general sense, the presence of metal ions affects the nucleation (or activation) and propagation of the self-assembly process regardless of the exact peptide sequence. Additionally, as evidenced by FIGS. 5A and 5B, the nucleation and propagation of the self-assembly process may be altered by modifying certain segments of the peptide. In this regard, another embodiment of the process includes the step of altering segments within a peptide to affect the nucleation and propagation of the self-assembly process. While FIGS. 5A and 5B show metal ions as specific nucleating elements and inhibiting elements, it should be appreciated that other substances may be used as a nucleating element or inhibiting element. For example, as discussed with reference to the structure of the peptides, any substance that binds to a residue to affect the structure may be used as a nucleating element or an inhibiting element. Additionally, the inhibiting element may affect any of the self-assembly pathways that are undergone by the peptide during the self-assembly process. In this regard, if the particular location and structure of the binding sites changes as a function of time, then different stages of the self-assembly process may be inhibited or activated by such controlling substances. As non-limiting examples, other nucleating or inhibiting elements may include other metal ions, small organic molecules, designed peptides and peptide analogs, nucleic acid analogs, or a combination of these elements.

As shown in FIGS. 3 and 4, since the metal ions likely bind at certain binding sites along the peptide, the rate of formation may be a function of the metal-ion-to-peptide concentration ratio. Thus, providing a greater metal-ion-to-peptide concentration ratio may more rapidly saturate the binding sites with the metal ions. In this regard, in another embodiment, changes in metal-ion-to-peptide concentration ratios may be altered to affect the resulting morphology. As non-limiting examples, a higher metal-ion-to-peptide ratio may be approximately 1.5 while a lower metal-ion-to-peptide ratio may be approximately 0.3. Also, since addition of metal ions also affects dielectric characteristics, in other embodiments, changes in the dielectric characteristics of the controlled environment may effect changes in morphology. While the addition of metal ions illustrates changes in dielectric characteristics, it should be appreciated that the dielectric characteristics may be changed by other known techniques.

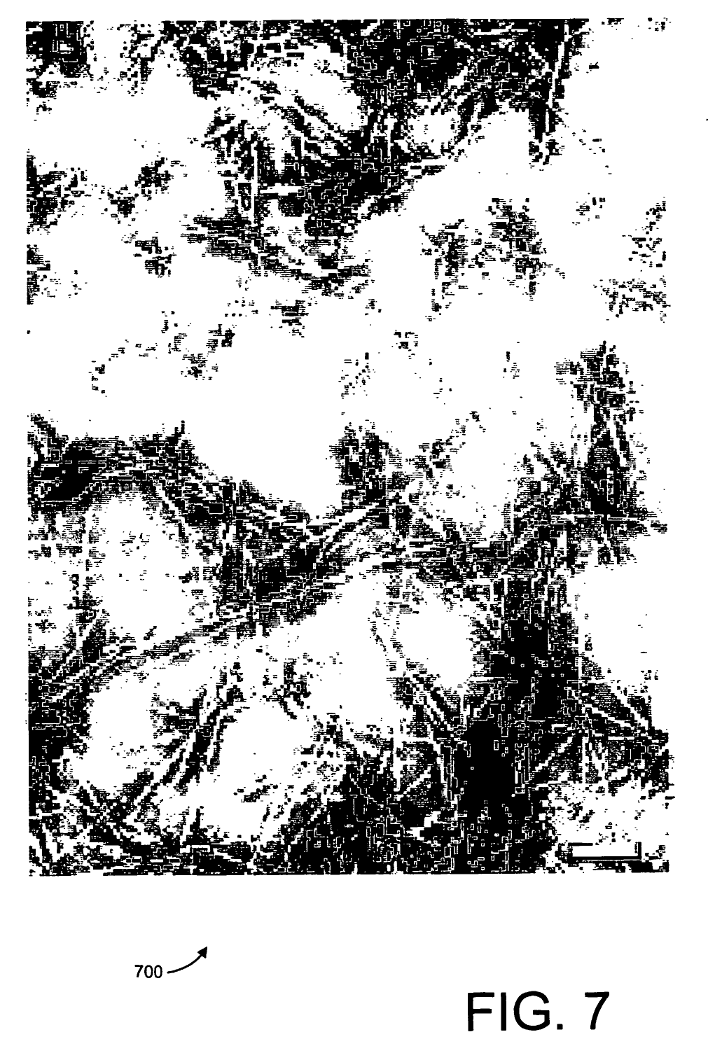

FIGS. 6 and 7 are diagrams showing different resulting fibers that are formed in the absence and presence of metal ions. Specifically, FIG. 6 shows the resulting morphology in the absence of ZnCl₂ at an approximate pH of 2. As discussed above, in the absence of zinc ions, and at a lower pH, the rate of assembly is relatively slow. Consequently, the slow formation of the self-assembled structures results in long heterogeneous fibers. Conversely, as shown in FIG. 7, in the presence of Zn⁺², the faster rate of assembly results in numerous short fibers. Thus, FIGS. 6 and 7 suggest that the presence of metal ions not only affects the rate of assembly (as shown in FIGS. 5A and 5B), but also affects the stability of the resulting structure.

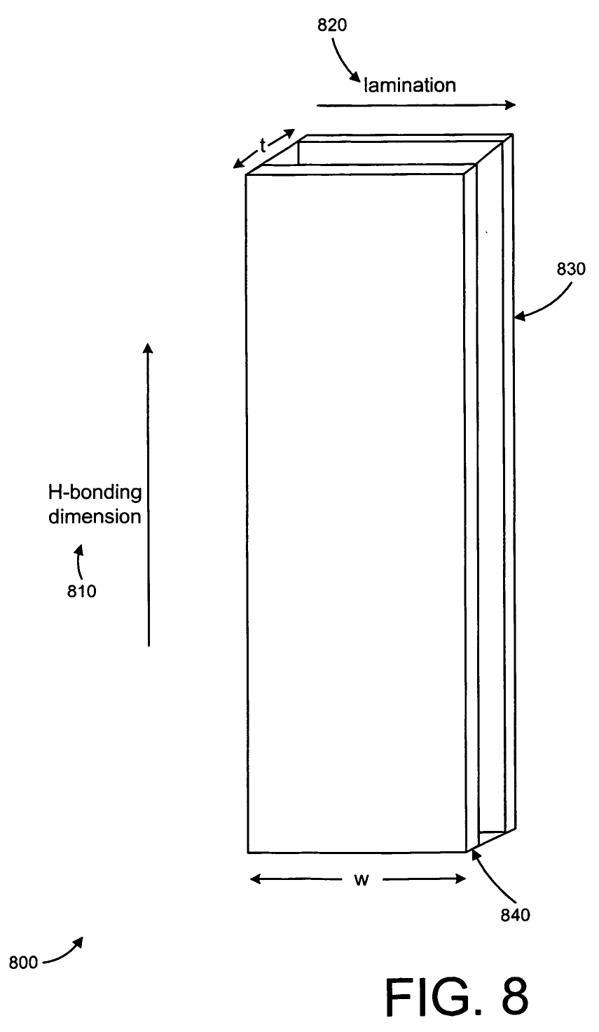

FIG. 8 is a diagram illustrating one embodiment of a structure as a rectangular bilayer 800 that is formed as an aggregate of fibril segments. Specifically, FIG. 8 shows a rectangular bilayer 800 formed using Aβ(16-22) (amino acid residues 16-22 of SEQ ID NO: 1), CH₃CO-KLVFFAE-NH₂. As shown in FIG. 8, the Aβ(16-22) (amino acid residues 16-22 of SEQ ID NO: 1) bilayer is approximately 130 nm wide by 4 nm thick, with each leaflet being composed of β-sheets. The corresponding backbone H-bond is shown along the long axis 810 of the rectangular bilayer 800, while the lamination is shown to progress along the 130-nm width of the rectangular bilayer 800. Since this rectangular bilayer 800 structure is described in greater detail in the paper “Exploiting Amyloid Fibril Lamination for Nanotube Self-Assembly,” further discussion of the rectangular bilayer 800 is omitted here. However, as further described below, the bilayer structure of FIG. 8 is used to further construct other architectures. In this regard, one embodiment of a self-assembling-peptide-based structure may be seen as a peptide bilayer similar to that shown in FIG. 8.

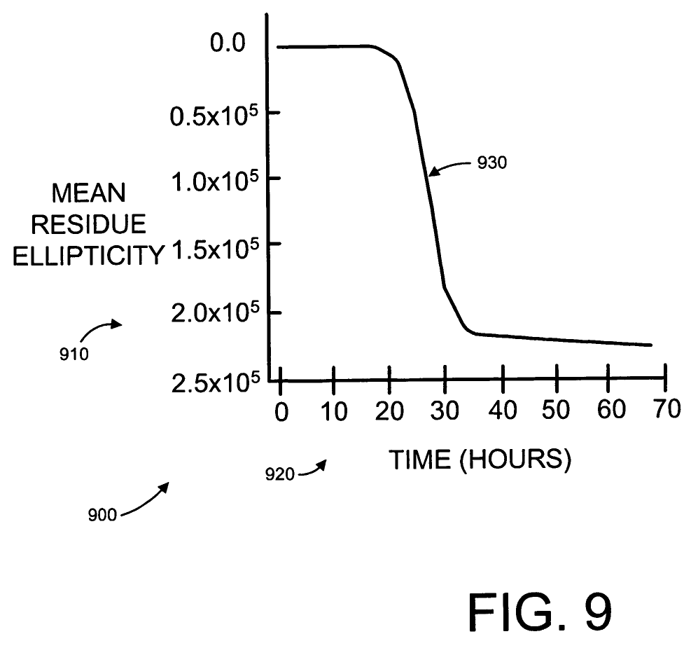

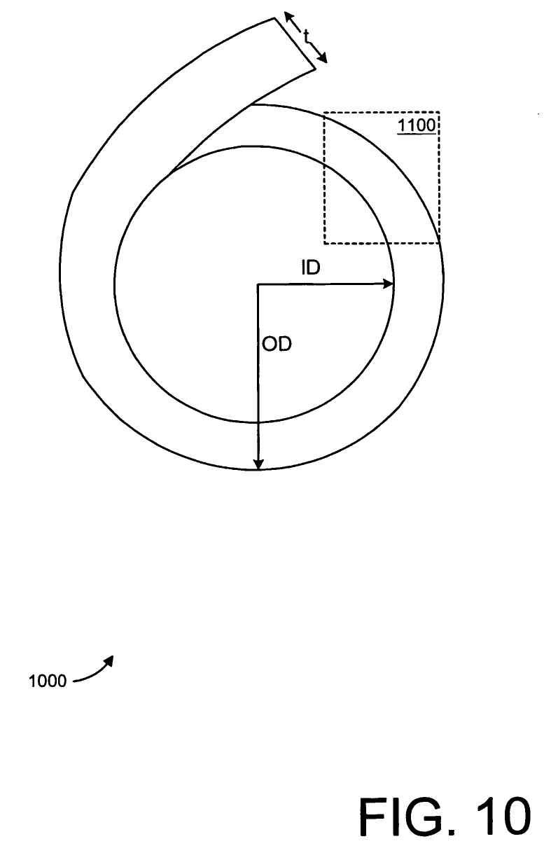

FIG. 9 is a graph 900 showing progression of mean residue ellipticity 930 over time, which is indicative of the structures being formed over time. The mean residue ellipticity is plotted on the y-axis 910 while the time is plotted on the x-axis 920 of the graph 900. As shown in FIG. 9, the mean residue ellipticity 930 shows that, after approximately 20 hours, a negative ellipticity developed, which suggests the formation of β-sheets. Within the following 10 hours, the ellipticity changed dramatically, thereby suggesting the formation of helical ribbons, which further progressed to nanotube structures. Since the progression of the structures is discussed in greater detail in the paper “Exploiting Amyloid Fibril Lamination for Nanotube Self-Assembly,” only a truncated discussion is presented herein. It is worthwhile to note, however, that the resulting peptide-based nanotube structure is more robust and stable than lipid-based nanotubes. Additionally, as shown in FIG. 9 and the paper “Exploiting Amyloid Fibril Lamination for Nanotube Self-Assembly,” the peptide-based nanotubes are generated using a thermodynamic process, rather than a high-energy kinetic process as those required to generate graphite-based nanotubes.

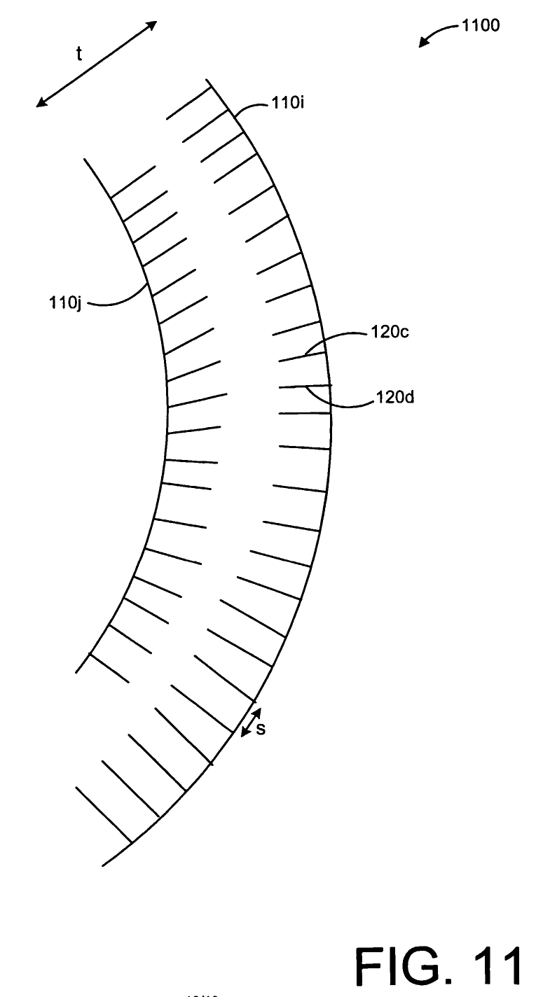

FIG. 10 is a diagram illustrating a top view of an example nanotube 1000 formed from Aβ(16-22) (amino acid residues 16-22 of SEQ ID NO: 1). As shown in FIG. 10, and also in the paper “Exploiting Amyloid Fibril Lamination for Nanotube Self-Assembly,” the nanotube 1000 has an inner radius of approximately 22 nm, an outer radius of approximately 26 nm, and a wall thickness (t) of approximately 4 nm. The wall thickness of approximately 4 nm is roughly twice the length of the Aβ(16-22) (amino acid residues 16-22 of SEQ ID NO: 1) peptide, which suggests that the wall of the nanotube 1000 is composed of a peptide bilayer similar to that shown in FIG. 8. This is shown in greater detail in FIG. 11.

FIG. 11 is an exploded view of a section of the nanotube 1000 defined by the broken lines 1100 in FIG. 10. As shown in FIG. 11 and the paper “Exploiting Amyloid Fibril Lamination for Nanotube Self-Assembly,” the Aβ(16-22) (amino acid residues 16-22 of SEQ ID NO: 1) peptide generates a bilayer that is approximately 4 nm thick. The bilayer of FIG. 11 is similar to the bilayer structure of FIG. 8 in that inner and outer surfaces of the bilayer are defined by β-sheets 110i, 110j, and each parallel β-strand 120c, 120d is separated by a fixed separation (s) defined by the backbone H-bond. For the Aβ(16-22) (amino acid residues 16-22 of SEQ ID NO: 1), the separation (s) is approximately 5 Å. Since the nanotube 1000 is discussed in greater detail in the paper “Exploiting Amyloid Fibril Lamination for Nanotube Self-Assembly,” only a truncated discussion of the nanotube is presented here. However it should be appreciated that the peptide-based nanotube 1000 results from a thermodynamic process, rather than a high-energy kinetic process that is required for generation of graphite-based nanotubes, which results in a relatively low overhead. Additionally, unlike lipid-based structures, the peptide-based nanotube 1000 is fairly rigid and robust due to the H-bonds that, in part, define the structure.

In addition to forming such structures, the paper “Exploiting Amyloid Fibril Lamination for Nanotube Self-Assembly” shows that the nanotubes melt (or become unstable) at higher temperatures (e.g., approximately 80 degrees Celsius). In this regard, for other embodiments, controlling temperatures (e.g., maintaining a temperature less than approximately 80 degrees Celsius) during the self-assembly process may be seen as one approach to controlling the morphology of a final self-assembled structure.

As described with reference to FIGS. 1 through 11, and in the paper “Metal Switch for Amyloid Formation: Insight into the Structure of the Nucleus,” the paper “Exploiting Amyloid Fibril Lamination for Nanotube Self-Assembly,” and other papers included in the above-identified provisional patent applications, the ability to manipulate the self-assembly process related to self-assembling peptides results in a novel approach to generating nanostructures. Additionally, by controlling the environment in which the self-assembling peptide undergoes the self-assembly process, the morphology of the resulting structures may be altered. Furthermore, given the mechanisms that underlie the assembly of self-assembling peptides, these processes may be activated and deactivated by controlling the environment in which the processes take place.

Although exemplary embodiments have been shown and described, it will be clear to those of ordinary skill in the art that a number of changes, modifications, or alterations may be made, none of which depart from the spirit of the present invention. For example, while specific peptides have been illustrated above, it should be appreciated that variants of those disclosed embodiments are also within the scope of this invention. These variants may be generated by adding, deleting, or substituting at least one amino acid, where the change may occur at the amino- or carboxy-terminal positions of the reference peptide sequence or anywhere between those terminal positions, interspersed either individually among the amino acids in the sequence or in one or more contiguous groups within the sequence, etc. Also, while not specifically discussed, it should be appreciated that other environmental factors, such as, for example, the media dielectric of the environment, may also be altered to effect morphological changes. Moreover, while nanotubes, peptide bilayers, helices, long and short fibers, β-sheets, β-strands, etc. have been described above, it should be appreciated that the other structures may also be generated using the processes described above. Furthermore, in the context of nanostructures described herein, long fibers are defined as any fiber having a fiber length that is greater than or equal to 500 nm, and short fibers are defined as those fibers having fiber lengths less than 500 nm. Additionally, while Aβ(10-21) (amino acid residues 10-21 of SEQ ID NO: 1), Aβ(10-21)H13Q (SEQ ID NO: 3), and Aβ(16-22) (amino acid residues 16-22 of SEQ ID NO: 1) have been explicitly discussed, it should be appreciated that the β-amyloid structure may be Aβ(16-21) (amino acid residues 16-21 of SEQ ID NO: 1), Aβ(10-35) (amino acid residues 10-35 of SEQ ID NO: 1), Aβ(10-21)E11N (SEQ ID NO: 2), Aβ(1-40) (amino acid residues 1-40 of SEQ ID NO: 1), Aβ(1-42) (SEQ ID NO: 1; ¹DAEFRHDSG¹⁰YEVHHQKLVFFAEDVGSNKGAIIGL³⁵MVGGVVI⁴²A), etc. All such changes, modifications, and alterations should therefore be seen as within the scope of the present invention.

“Polypeptide” refers to any peptide or protein comprising two or more amino acids joined to each other by peptide bonds or modified peptide bonds, (i.e., peptide isosteres). “Polypeptide” refers to both short chains (commonly referred to as peptides, oligopeptides, or oligomers) and to longer chains (generally referred to as proteins). “Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (Proteins—Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter, et al., Meth Enzymol, 182: 626-646, 1990, and Rattan, et al., Ann NY Acad. Sci., 663:48-62, 1992).

“Variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polypeptide may be a naturally occurring such as an allelic variant, or it may be a variant that is not known to occur naturally. Non-naturally occurring variants of polypeptides may be made by mutagenesis techniques or by direct synthesis.

“Identity,” as known in the art, is a relationship between two or more polypeptide sequences as determined by comparing the sequences. In the art, “identity” also means the degree of sequence relatedness between polypeptide sequences, as the case may be, as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453,1970) algorithm (e.g., NBLAST, and XBLAST). The default parameters are used to determine the identity for the polypeptides of the present invention.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

Embodiments of the present invention also provide for amyloid polypeptides that are substantially homologous to the amyloid polypeptides of SEQ ID NO: 1. The term “substantially homologous” is used herein to denote polypeptides having about 50%, about 60%, about 70%, about 80%, about 90%, and preferably about 95% sequence identity to the sequences shown in SEQ ID NO: 1. Percent sequence identity is determined by conventional methods as discussed above.

In general, homologous polypeptides are characterized as having one or more amino acid substitutions, deletions, and/or additions. These changes are preferably of a minor nature, that is conservative amino acid substitutions and other substitutions that do not significantly affect the activity of the polypeptide; small substitutions, typically of one to about six amino acids; and small amino- or carboxyl-terminal extensions, such as an amino-terminal methionine residue, a small linker peptide of up to about 2-6 residues, or an affinity tag. Homologous polypeptides comprising affinity tags can further comprise a proteolytic cleavage site between the homologous polypeptide and the affinity tag.

In addition, embodiments of the present invention include polypeptides having one or more “conservative amino acid substitutions,” compared with the amyloid polypeptide of SEQ ID NO: 1. Conservative amino acid substitutions can be based upon the chemical properties of the amino acids. That is, variants can be obtained that contain one or more amino acid substitutions of SEQ ID NO: 1, in which an alkyl amino acid is substituted for an alkyl amino acid in a amyloid polypeptide, an aromatic amino acid is substituted for an aromatic amino acid in a amyloid polypeptide, a sulfur-containing amino acid is substituted for a sulfur-containing amino acid in a amyloid polypeptide, a hydroxy-containing amino acid is substituted for a hydroxy-containing amino acid in a amyloid polypeptide, an acidic amino acid is substituted for an acidic amino acid in a amyloid polypeptide, a basic amino acid is substituted for a basic amino acid in a amyloid polypeptide, or a dibasic monocarboxylic amino acid is substituted for a dibasic monocarboxylic amino acid in a amyloid polypeptide.

Amyloid polypeptides having conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine.

A limited number (i.e., less than 6) of non-conservative amino acids, amino acids that are not encoded by the genetic code, non-naturally occurring amino acids, and unnatural amino acids may be substituted for amyloid polypeptide amino acid residues.

Using the methods discussed herein, one of ordinary skill in the art can identify and/or prepare a variety of amyloid polypeptide fragments or variants of SEQ ID NO: 1 that retain the functional properties of the amyloid polypeptide.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions are provided in Table 1.

TABLE 1

| Characteristic | Amino Acid |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

1. A process for controlling self-assembly of self-assembling-peptide-based structures, the process comprising:
(A) providing a controlled environment by:
(A1) controlling content of metal ions within the controlled environment, the metal ions being selected from a group consisting of:
(A1a) zinc ions; and
(A1b) copper ions;
(A2) controlling the acidity of the controlled environment, the acidity being within the range of approximately pH 2.0 to approximately pH 7.4;
(A3) controlling the temperature of the controlled environment, the temperature being less than approximately 80 degrees Celsius;
(A4) controlling the dielectric characteristics of the controlled environment;
(A5) controlling a metal-ion-to-peptide concentration ratio in the controlled environment, the metal-ion-to-peptide concentration ratio being within the range of approximately 0.3 to approximately 1.5;
(B) placing segments of β-amyloids in the controlled environment to generate a self-assembling structure,
(B 1) wherein the self-assembling structure is selected from a group consisting of:
(B1a) a long fiber having a fiber length not less than approximately 500 nm;
(B1b) a short fiber having a fiber length less than approximately 500 nm;
(B1c) a helical structure;
(B1d) a twisted ribbon structure;
(B1e) a fibrillar structure;
(B1f) a peptide bilayer; and
(B1g) a nanotube;
(B2) wherein the segment of the β-amyloid is selected from the group consisting of:
(B2a) amino acid residues 10-21 of SEQ ID NO: 1 (Aβ(10-21));
(B2b) SEQ ID NO: 2 (Aβ(10-21)E11N);
(B2c) SEQ ID NO: 3 (Aβ(10-21)H13Q);
(B2d) amino acid residues 10-35 of SEQ ID NO: 1 (Aβ(10-35));
(B2e) amino acid residues 16-21 of SEQ ID NO: 1 (Aβ(16-21));
(B2f) amino acid residues 16-22 of SEQ ID NO: 1 (Aβ(16-22));
(B2g) amino acid residues 18-28 of SEQ ID NO: 1 (Aβ(18-28));
(B2h) amino acid residues 1-40 of SEQ ID NO: 1 (Aβ(1-40));
(B2i) SEQ ID NO: 1 (Aβ(1-42)).

2. A process for controlling self-assembly of peptide-based structures, the process comprising:
providing a controlled environment, the controlled environment being adapted to redirect a self-assembly process, the self-assembly process being associated with a self-assembling peptide; and
generating a self-assembling-peptide-based structure by placing the self-assembling peptide in the controlled environment.

3. The process of claim 2, wherein the step of providing the controlled environment comprises:
activating the self-assembly process by introducing a nucleating element.

4. The process of claim 3, wherein the step of activating the self-assembly process comprises:
introducing a metal ion.

5. The process of claim 2, wherein the step of providing the controlled environment comprises:
inhibiting a self-assembly pathway by introducing an inhibiting element.

6. The process of claim 5, wherein the step of inhibiting the self-assembly pathway comprises:
introducing a metal ion.

7. The process of claim 2, wherein the step of providing the controlled environment comprises a step selected from the group consisting of:
controlling content of nucleating elements within the controlled environment; and
controlling content of inhibiting elements within the controlled environment.

8. The process of claim 7, wherein the step of controlling the content of nucleating elements comprises:
controlling content of zinc ions within the controlled environment.

9. The process of claim 7, wherein the step of controlling the content of inhibiting elements comprises:
controlling content of copper ions within the controlled environment.

10. The process of claim 2, wherein the step of providing the controlled environment comprises a step selected from the group consisting of:
controlling a nucleating-element-to-peptide concentration ratio within the controlled environment; and
controlling an inhibiting-element-to-peptide concentration ratio within the controlled environment.

11. The process of claim 2, wherein the step of providing the controlled environment comprises:
controlling the acidity of the controlled environment.

12. The process of claim 2, wherein the step of providing the controlled environment comprises:
controlling the temperature of the controlled environment.

13. The process of claim 2, wherein the step of providing the controlled environment comprises:
controlling the dielectric characteristics of the controlled environment.

14. The process of claim 2, wherein the step of generating the self-assembling structure comprises:
generating a long fiber having a fiber length not less than approximately 500 nm.

15. The process of claim 2, wherein the step of generating the self-assembling structure comprises:
generating a short fiber having a fiber length less than approximately 500 nm.

16. The process of claim 2, wherein the step of generating the self-assembling structure comprises:
generating a helical structure.

17. The process of claim 2, wherein the step of generating the self-assembling structure comprises:
generating a peptide bilayer.

18. The process of claim 2, wherein the step of generating the self-assembling structure comprises:
generating a nanotube.

19. The process of claim 2, wherein the step of generating the self-assembling structure comprises:
placing a segment of a β-amyloid in the controlled environment, wherein the segment of the β-amyloid is selected from a group consisting of:
amino acid residues 10-21 of SEQ ID NO: 1) (Aβ(10-21) and variants thereof;
SEQ ID NO: 2 (Aβ(10-21)E11N) and variants thereof;
SEQ ID NO: 3 (Aβ(10-21)H13Q) and variants thereof;
amino acid residues 16-21 of SEQ ID NO: 1 (Aβ(16-21)) and variants thereof;
amino acid residues 16-22 of SEQ ID NO: 1 (Aβ(16-22)) and variants thereof.

20. The process of claim 2, wherein the step of generating the self-assembling structure comprises:
placing a segment of a β-amyloid in the controlled environment, wherein the segment of the β-amyloid is selected from a group consisting of:
amino acid residues 10-35 of SEQ ID NO: 1 (Aβ(10-35)) and variants thereof;
amino acid residues 18-28 of SEQ ID NO: 1 (Aβ(18-28)) and variants thereof;
amino acid residues 1-40 of SEQ ID NO: 1 (Aβ(1-40)) and variants thereof; and
SEQ ID NO: 1 (Aβ(1-42)) and variants thereof.

21. A process for controlling self-assembly of self-assembling-peptide-based structures, the process comprising:
placing a self-assembling peptide in a controlled environment;
controlling initiation of a self-assembly process, the self-assembly process being associated with the self-assembling peptide; and
controlling propagation of the self-assembly process.

22. The process of claim 21, wherein the step of placing the self-assembling peptide in the controlled environment comprises:
placing a segment of a β-amyloid in the controlled environment, wherein the segment of the β-amyloid is selected from a group consisting of:
amino acid residues 10-21 of SEQ ID NO: 1 (Aβ(10-21) and variants thereof;
SEQ ID NO: 2 (Aβ(10-21)E11N) and variants thereof;
SEQ ID NO: 3 (Aβ(10-21)H13Q) and variants thereof;
amino acid residues 16-21 of SEQ ID NO: 1 (Aβ(16-21)) and variants thereof;
amino acid residues 16-22 of SEQ ID NO: 1 (Aβ(16-22)) and variants thereof.

23. The process of claim 21, wherein the step of placing the self-assembling peptide in the controlled environment comprises:
placing a segment of a β-amyloid in the controlled environment, wherein the segment of the β-amyloid is selected from a group consisting of:
amino acid residues 10-35 of SEQ ID NO: 1 (Aβ(10-35)) and variants thereof;
amino acid residues 18-28 of SEQ ID NO: 1 (Aβ(18-28)) and variants thereof;
amino acid residues 1-40 of SEQ ID NO: 1 (Aβ(1-40)) and variants thereof;
SEQ ID NO: 1 (Aβ(1-42)) and variants thereof.

24. The process of claim 21, wherein the step of controlling initiation of the self-assembly process comprises:
activating the self-assembly process by adding a nucleating element.

25. The process of claim 21, wherein the step of controlling initiation of the self-assembly process comprises:
inhibiting the self-assembly process by adding an inhibiting element.

26. The process of claim 21, wherein the step of controlling initiation of the self-assembly process comprises a step selected from the group consisting of:
controlling content of nucleating elements within the controlled environment; and
controlling content of inhibiting elements within the controlled environment.

27. The process of claim 21, wherein the step of controlling initiation of the self-assembly process comprises a step selected from the group consisting of:
controlling a nucleation-element-to-peptide concentration ratio in the controlled environment; and
controlling an inhibiting-element-to-peptide concentration ratio in the controlled environment.

28. The process of claim 21, wherein the step of controlling initiation of the self-assembly process comprises:
controlling the temperature of the controlled environment.

29. The process of claim 21, wherein the step of controlling propagation of the self-assembly process comprises:
controlling content of metal ions within the controlled environment.

30. The process of claim 21, wherein the step of controlling propagation of the self-assembly process comprises:
controlling a metal-ion-to-peptide concentration ratio in the controlled environment.

31. The process of claim 21, wherein the step of controlling propagation of the self-assembly process comprises:
controlling the temperature of the controlled environment.

32. A self-assembling-peptide-based structure comprising:
segments of a β-amyloid, the segments being selected from a group consisting of:
amino acid residues 10-21 of SEQ ID NO: 1 (Aβ(10-21);
SEQ ID NO: 2 (Aβ(10-21)E11N);
SEQ ID NO: 3 (Aβ(10-21)H13Q);
amino acid residues 16-21 of SEQ ID NO: 1 (Aβ(16-21));
amino acid residues 16-22 of SEQ ID NO: 1 (Aβ(16-22));
amino acid residues 10-21 of SEQ ID NO: 1 (Aβ(10-21) with a conservative amino acid substitution;
SEQ ID NO: 2 (Aβ(10-21)E11N) with a conservative amino acid substitution;
SEQ ID NO: 3 (Aβ(10-21)H13Q) with a conservative amino acid substitution;
amino acid residues 16-21 of SEQ ID NO: 1 (Aβ(16-21)) with a conservative amino acid substitution; and
amino acid residues 16-22 of SEQ ID NO: 1 (Aβ(16-22)) with a conservative amino acid substitution; and
hydrogen bonds formed between the segments of the β-amyloid.

33. The self-assembling-peptide-based structure of claim 32, wherein the structure is a long fiber having a fiber length not less than approximately 500 nm.

34. The self-assembling-peptide-based structure of claim 32, wherein the structure is a short fiber having a fiber length less than approximately 500 nm.

35. The self-assembling-peptide-based structure of claim 32, wherein the structure is a peptide bilayer.

36. The self-assembling-peptide-based structure of claim 32, wherein the structure is a fibrillar structure.

37. The self-assembling-peptide-based structure of claim 32, wherein the structure is a helical structure.

38. The self-assembling-peptide-based structure of claim 32, wherein the structure is a twisted ribbon structure.

39. The self-assembling-peptide-based structure of claim 32, wherein the structure is a nanotube.

40. The self-assembling-peptide-based structure of claim 39, wherein the nanotube comprises:
a wall thickness of approximately 4 nm; and
an outer diameter between approximately 50 nm and approximately 100 nm.

41. The self-assembling-peptide-based structure of claim 32, wherein the structure is a peptide bilayer having:
a thickness of approximately 4 nm; and
a width of approximately 130 nm.

42. A self-assembling-peptide-based structure comprising:
self-assembling peptides; and
hydrogen bonds formed between self-assembling peptides to form a nanotube.

43. The self-assembling-peptide-based structure of claim 42, wherein the nanotube comprises:
a wall thickness of approximately 4 nm; and
an outer diameter between approximately 50 nm and approximately 100 nm.
